(19) United States
(12) Patent Application Publication
(10) Pub. No.: US 2005/0164979 A1
(43) Pub. Date: Jul. 28, 2005

(54) Pharmaceutical composition for ophthalmological and rhinological application

(76) Inventors: Dorothea Gross, Ingbert (DE); Frank Holzer, Ingbert (DE)

Correspondence Address: Carella Byrne Bain Gilfillan;Cecchi Stewart & Olstein, 6 Becker Farm Road, Roseland, NJ, 07068, US

(21) Appl. No.: 10/496,456

(22) PCT Filed: Dec. 11, 2002
(86) PCT No.: PCT/DE02/45217

(30) Foreign Application Priority Data
Dec. 12, 2001 (DE) 1016110.2-41

(51) Int. Cl. A61K 31/728; A61K 31/195

(52) U.S. Cl. 514/54; 514/561

(57) ABSTRACT

The invention concerns a pharmaceutical composition which contains at least panthenol and/or pantothenic acid and hyaluronic acid and/or hyaluronate and optionally additionally pharmaceutical auxiliary agents. The invention further concerns the use of the pharmaceutical composition for the treatment of ophthalmological and/or rhinological malfunctions.

The invention concerns a pharmaceutical composition and the use thereof for the treatment of ophthalmological and rhinological malfunctions.

The “dry eye” syndrome is also referred to as the Sicca syndrome or also as the Sicca symptoms. The “dry eye” syndrome, the symptoms of which involve inter alia burning, scratchiness, and a gritty feeling in the eye as well as blurred vision is to be attributed to functional disturbances in the tear film.

The “dry eye” syndrome can also be attributed to a reduced flow of tears, which can involve various pathological causes. The reduced flow of tears can result in the formation of only an inadequate or no tear film on the surface of the eye. The tear film acts inter alia as a slip or lubricating agent between the eyelid and the surface of the eye. As a result of the absence of or inadequate tear film, the epithelial layers can suffer from considerable trauma.

The cause of those functional disturbances is also for example environmental influences which give rise to allergies, such as for example pollution or the effect of ozone. In particular ozone pollution which rises in Summer months can not only give rise to a disturbance in tear production but can also cause a disturbance in the physiological tear film. For example hyaluronic acid and proteins contained in natural tear film are destroyed by the effect of ozone. It has further been found that the Sicca syndrome is frequently linked to a contact allergy caused by cosmetics on the eye.

It is known from Spektrum Augenheilkunde (1998) 3/4: 174-176 that hypoosmolar sodium hyaluronate drops can be used for therapy for the “dry eye” syndrome. In that case sodium hyaluronate of a molecular weight of 5,000,000 Daltons was used.

It is further known from Spektrum Augenheilkunde (1995) 9/5: 215-217 that bacterially synthesised hyaluronate can be used for treating the “dry eye” syndrome. It is known from Jpn. J. Ophthalmol. (1996) 40: 62-65 that the improvement in tear film stability is achieved by means of sodium hyaluronate eye drops which contain at least 0.1% of sodium hyaluronate.

It is known from Klinische Monatsblätter für Augenheilkunde (1996) 209; 84-88 that dexpanthenol-bearing eye drops improve the cornea-epithelial barrier function which is disturbed by virtue of an insufficient tear film.

The use of panthenol is further known for the treatment of cauterisation effects, burns and ray damage to the skin as well as for the treatment of inflammation of the eyes.

The object of the present invention is to provide a pharmaceutical composition, which permits better therapy of diseases of the eye which in particular involve functional disturbances to the tear film or a reduced flow of tears.

A further object of the invention is to provide a pharmaceutical composition which permits a treatment of dry mucous membrane of the nose.

That object is attained by the provision of a pharmaceutical composition which includes at least panthenol and/or pantothenic acid and hyaluronic acid and/or hyaluronate and optionally additionally pharmaceutical auxiliary agents.

The term “pharmaceutical auxiliary agents” is used to denote solvents, dissolving aids, dissolving accelerators, salt-forming agents, salts, buffer substances, viscosity- and consistency-influencing agents, gel-forming agents, emulsifiers, solubilisers, wetting agents, spreading agents, antioxidants, preserving agents, filling and carrier substances and so forth.

Panthenol, that is to say (R, S)-2,4-dihydroxy-N-(3-hydroxypropyl)-3,3-dimethylbutyramide, which is also referred to as pantothenol or pantothenyl alcohol, is known as an epithelialisation agent for the skin. Panthenol is the alcoholic analog of pantothenic acid and by virtue of the intermediate conversion has the same biological effectiveness as pantothenic acid.

It has been found that the pharmaceutical composition according to the invention can be used both for the therapy of ophthalmological malfunctions and also for the therapy of rhinological malfunctions.

The pharmaceutical composition is particularly well suited to the treatment of dried-out, dry or chronically dry mucous membrane of the nose.

The mucous membrane of the nose can suffer from drying out for example in air-conditioned spaces or vehicles or for example in excessively dry spaces and rooms which are overheated in winter. In an unnaturally dry environment the mucous membrane of the nose can no longer fulfil its task of pre-moistening the inhaled air. The mucous membrane of the nose then swells shut as when suffering from a head cold. Consequently secretion is no longer formed but dry crusts are formed, which easily result in bleeding cracks in the mucous membrane in the nose. A nosebleed can possibly also occur.

Drying-out of the mucous membrane of the nose is also promoted for example by dust at the workplace or by pollutants such as cigarette smoke, formaldehyde, sulphur oxides, nitrogen oxides and the so forth. In addition drying-out of the mucous membrane of the nose can also be attributed to nasal septum curvature or inflammation of the mucous membrane during a cold or an allergy. Dry mucous membrane cells die off. In addition pathogens can pass into the body by way of the dried-out mucous membrane. Extreme cases can involve hole formation in the septum because the mucous membrane cells which have died off no longer adequately supply the cartilage tissue therebeneath.

Drying-out of the mucous membrane of the nose can also be due to medicational influences such as for example on-going use of swelling-reducing cold agents.

A dried-out mucous membrane of the nose can further result in complaints, in particular when breathing, and thus troubles such as for example difficulty in going to sleep or snoring while asleep.

The pharmaceutical composition according to the invention extremely advantageously has a dual effect. On the one hand hyaluronic acid or the salts thereof have a high water binding capacity which counteracts or counteract drying-out of the mucous membrane of the nose. On the other hand panthenol and/or pantothenic acid provide for faster healing of wounds when injuries have already occurred to the nasal mucous membrane, for example due to mechanical removal of the crusts formed, as for example by ‘nose boring’.

The composition according to the invention is thus used in the case of drying-out of the mucous membrane of the nose, caused by environmental factors, and also in relation to pathologically induced drying-out of the nasal mucous membrane.

The simultaneous supply of moisture to the mucous membrane of the nose and the prevention of fast drying-out of the mucous membrane, and also the improved wound healing effect, give rise to a rapid reduction in swelling of the mucous membrane, reduced itching and a ‘clear nose’ through which the person in question can breathe again.

The composition according to the invention results in fast healing and alleviation of the troubles in the case of people with dry and/or dried-out mucous membrane of the nose.

The pharmaceutical composition according to the invention can be particularly advantageously used in the treatment of chronic rhinitis, rhinitis sicca, rhinitis sicca anterior and hybrid forms thereof.

The further information hereinafter is set forth essentially in relation to the ophthalmological use of the pharmaceutical application. This information however correspondingly applies to rhinological use. For example, in the case of a formulation for rhinological application, the same forms of administration and the same compositions can be used.

The inventors of the present invention further surprisingly found that, with the simultaneous application of panthenol and/or pantothenic acid and hyaluronic acid and/or hyaluronate to the eye or on the surface of the eye, a synergistic effect occurs in regard to the treatment of ophthalmological diseases which are linked to functional disturbances of the tear film or an inadequately formed tear film.

In particular it has been found that accelerated epithelialisation of a cornea damaged by virtue of an insufficient tear film occurs with topical application of the pharmaceutical composition according to the invention.

Preferably the pharmaceutical composition according to the invention is prepared, in the treatment of ophthalmological malfunctions, in the form of eye drops, eye solutions, eye lotions, eye sprays, eye ointments or eye tablets for topical application to the eye or the surface of the eye.

In the case of rhinological use the pharmaceutical composition is preferably prepared in the form of nasal sprays, nose drops or nose ointments.

To produce a nose or eye ointment the hyaluronic acid and/or hyaluronate and at least panthenol and/or pantothenic acid can be introduced for example into a mixture of viscous paraffin and white Vaseline. In addition for example low-viscosity paraffin or wool wax can also be used in ointments.

Preferably the pharmaceutical composition is prepared in the form of a nose or eye spray or in the form of nose or eye drops. In that respect generally the hyaluronic acid and/or hyaluronate and the panthenol and/or pantothenic acid are dissolved in aqueous solutions.

In that respect, in accordance with a preferred embodiment for ophthalmological use, the aqueous solutions are isotonic solutions, with respect to the tear fluid. In the case of isotonic solutions osmolarity is approximately 300 mOsm/l. In accordance with a further preferred embodiment the pharmaceutical composition according to the invention is hypoosmolar. In that case osmolarity can be for example about 160-180 mOsm/l. A hypoosmolar solution is used in particular when an abnormally high level of osmolarity of a tear film in a patient with dry eyes has to be compensated.

Sodium chloride, boric acid etc. are used for isotonisation of the aqueous solution. The pH-value of the aqueous solution is in a range of pH 6 to 9, preferably pH 6.5 to 8.5, further preferably pH 7.4. Buffer solutions such as for example phosphate buffer, acetate buffer, acetate-borate buffer, citrate buffer and borate buffer are used to adjust the pH-value.

In the case of nose drops or nasal sprays for the treatment of dried-out or dry nasal mucous membrane the active substances, that is to say pantothenic acid and/or panthenol and hyaluronic acid and/or hyaluronate(s) are put in a suitable, preferably isotonic, medium. Preferably sorbitol is used for isotonisation purposes. It is however also possible to use other media such as for example physiological saline solution.

As discussed hereinbefore with reference to the rhinological application of the pharmaceutical composition, hyaluronic acid or hyaluronates thereof has or have a high water binding capacity. That water binding capacity advantageously provides that the eye is supplied with moisture or drying-out of the eye is counteracted. In addition hyaluronic acid or hyaluronates thereof also acts or act as a viscosity regulator.

In the present case the term viscosity regulator is used to denote substances which are pharmacologically compatible and have a viscosity-increasing effect. Preferably the viscosity regulators which can be further used have a viscoelastic behaviour.

The viscosity-increasing effect extremely advantageously provides that the pharmaceutical composition applied to the surface of the eye or the nasal mucous membrane enjoys an increased residence time thereat and does not immediately flow away again from the surface of the eye or the nasal mucous membrane.

Besides the hyaluronic acid or hyaluronate it is additionally also possible to use chondroitin sulphate, polyacrylamide, polyacrylic acid, polyacrylic resins, polyethylene glycol, cellulose derivatives, polyvinyl alcohol, polyvinyl pyrrolidone or mixtures thereof as viscosity regulators.

In accordance with a preferred embodiment, besides hyaluronic acid and/or hyaluronate, no further viscosity regulator is used.

The hyaluronic acid or the hyaluronate can be isolated from the vitreous humour of a bovine eye but also from cockscombs. In addition hyaluronic acid or hyaluronate can also be produced in bacterial strains in pharmaceutical quality.

For example potassium, sodium, calcium and/or magnesium hyaluronates can be used as salts of hyaluronic acid.

The hyaluronate sodium hyaluronate is particularly preferred.

Hyaluronic acid is inter alia a constituent part of the vitreous humour of the eye and in that respect does not represent a compound which is foreign to the human organism. For that reason hyaluronic acid is very well compatible, from the immunological point of view.

Extremely advantageously hyaluronic acid or hyaluronate enjoys a structural similarity with mucin. Mucin forms the lowermost layer of the three-layer tear film and provides for optimum wetting of the cornea and conjunctiva epithelia.

Hyaluronic acid and/or hyaluronate thus imitates the mucous phase of the tear film and prolongs on the one hand the residence time on the eye and improves wettability of the eye. Imitation of the mucous phase on the other hand also causes a reduction in friction between the eye and the eyelid and thus a marked reduction in mechanical irritation of the eye.

The use of hyaluronic acid or hyaluronates in the pharmaceutical composition produced in accordance with the use of this invention is extremely advantageous in particular in terms of disturbance to wetting of the eye, that is to say in the case of what is referred to as “dry eye”, and for the treatment of epithelium lesions which result from disturbances to wetting of the eye.

Aqueous sodium hyaluronate solutions are fluids with non-Newtonian flow properties. By virtue of that physical property, aqueous sodium hyaluronate solutions are excellently well suited as slip and lubricating agents with a good cling effect and a prolonged residence time on the conjunctival and corneal epithelia, without adversely affecting visual efficiency. A concentration of 0.1% by weight of sodium hyaluronate in the composition according to the invention considerably improves the subjective feeling of the patients, which is important when treating dry eyes.

The non-Newtonian flow behaviour of the hyaluronic acid provides a property which is excellent for use on the eye, namely that viscosity decreases with increasing shearing rate.

After application of the pharmaceutical composition according to the invention to the cornea of the eye, a shearing stress is applied to the pharmaceutical composition by way of the blinking movement of the eyelid, whereby the initially increased viscosity is reduced. The viscosity is reduced due to the blinking movement of the eyelid so that a uniform film is formed on the surface of the eye. After the blink the viscosity increases so that the film adheres firmly to the surface of the eye.

The non-Newtonian flow properties of the pharmaceutical composition according to the invention, which are further produced by hyaluronic acid or hyaluronate in prepared solutions, gels, pastes or ointments, and the structural similarity thereof with mucin, besides an excellent slip and lubricating effect, also afford excellent adhesion to the cornea of the eye. Mechanical irritation of the eye which occurs with the Sicca syndrome is greatly reduced or eliminated. In addition, the fact that adhesion of the pharmaceutical composition on the cornea of the eye is improved by virtue of the anti-Newtonian flow properties provides for faster healing of epithelium lesions.

In addition sodium hyaluronate-bearing eye drops exhibit properties such as to promote healing of wounds on the epithelia of the eye, in animal testing. It was found that hyaluronic acid or sodium hyaluronate, in dependence on concentration, promoted the migration of epithelium cells and thus wound healing. A 0.1% by weight sodium hyaluronate solution implemented increased epithelium cell migration in the case of cornea epithelium cells of rabbits.

Hyaluronic acid or sodium hyaluronate also caused faster and better wound healing, that is to say which takes place with less scarring, in the event of injury to the cornea epithelium or in the case of cauterisation of the cornea.

The precise operative mechanism involved in the promotion of wound healing by hyaluronic acid is still unexplained. While an influence on the circulation of blood through the surrounding cells appears to be less probable, there are various pointers to an effect on cells which play a part in the inflammation process.

Finally, in dependence on dose, hyaluronic acid exhibits a protective action in relation to damage to cells by oxygen radicals. Free oxygen radicals slow down the wound healing process and thus play a crucial role in the inflammation situation.

The anti-inflammatory effect of hyaluronic acid or hyaluronate and the protection afforded by hyaluronic acid or hyaluronate from the harmful effect of oxygen radicals co-operate in synergistic relationship with the action of the panthenol or pantothenic acid in the pharmaceutical composition in accordance with the invention.

In accordance with a further preferred embodiment the hyaluronic acid and/or hyaluronate are of a molecular weight which is in a range of about 50,000 to about 10,000,000 Daltons, preferably from about 250,000 to about 5,000,000 Daltons. Particularly preferably the molecular weight of the hyaluronic acid or the hyaluronate is from 500,000 to 4,000,000 Daltons. Very preferably the hyaluronic acid or the hyaluronate is of a molecular weight of about 1,500,000 to 3,500,000 Daltons.

The high molecular weight of the hyaluronic acid or the hyaluronate used such as for example sodium hyaluronate provides for a high level of viscoelasticity at a low level of concentration. The molecule chains are present in the solution in a random arrangement in a tangled configuration. Under the influence of the shearing forces exerted by the movement of the eyelid, the macromolecules are oriented in substantially parallel relationship. That change in the three-dimensional structure under the influence of shearing forces is thought to be crucial for the excellent viscoelastic properties.

Upon lid opening the substance covers over the surface of the cornea and, by virtue of the high water binding capacity of hyaluronate, also represents protection against evaporation. That is advantageous both in relation to the “dry eye” syndrome which involves a reduction in the amount of tear fluid in the eye, and also in the treatment of a dried-out or dry nasal mucous membrane.

In accordance with a further preferred embodiment the amount of hyaluronic acid and/or the amount of hyaluronate is about 0.005% by weight to about 5% by weight, preferably about 0.01% by weight to about 1% by weight, in each case in relation to the total weight of the pharmaceutical composition.

Particularly preferably the amount of hyaluronic acid and/or the amount of hyaluronate is about 0.05% by weight to about 0.5% by weight, with respect to the total weight of the pharmaceutical formulation.

Extremely advantageously hyaluronic acid and hyaluronates respectively has or have the property of binding water. That property of binding water is particularly advantageous in regard to treatment of the Sicca syndrome as unwanted drying-out of the cornea of the eye is counteracted. Levels of concentration of 0.1% by weight to 0.3% by weight with respect to the total weight of the pharmaceutical formulation, hyaluronic acid and/or hyaluronate, have proven to be highly satisfactory.

A further diagnostic parameter in regard to diagnosis of the “dry eye” syndrome is the tear film tearing time which makes it possible to provide information about the quality of the tear fluid. In that case for example the tear film is dyed with fluorescein and the patient is then asked to keep the eyes open as long as possible without a blink reflex. A slit lamp is then used to establish when the tear film tears open for the first time. If the period of time is less than 10 seconds, there is the suspicion of the “dry eye” syndrome. In that respect hyaluronic acid at a concentration of 0.1% by weight to 0.3% by weight has proven to be highly effective in regard to prolonging the tear film tearing time.

The simultaneous action of panthenol and/or pantothenic acid and the provision of an artificial tear film leads to a synergistic effect which permits faster therapy of epithelium lesions, in particular in the “dry eye” syndrome.

It has surprisingly been found that the joint application of panthenol and/or pantothenic acid and hyaluronic acid and/or hyaluronate leads to rapid epithelialisation. At the same time extremely unpleasant itching which occurs when epithelium lesions on the eye are involved is quickly alleviated.

The pharmaceutical composition according to the invention is accordingly a drug combination.

It has been found that the hyaluronic acid and/or hyaluronate, by virtue of the non-Newtonian flow behaviour, have very suitable viscoelastic properties for use on the surface of the eye. The non-Newtonian flow behaviour delays the draining away of the pharmaceutical composition applied to the eye and thus prolongs the contact with the cornea of the eye. Accordingly the panthenol and/or pantothenic acid can be kept on the cornea over a longer period of time, for example at least 30 minutes to at least 60 minutes.

Caused by the viscoelastic properties of hyaluronic acid or hyaluronate, the panthenol and/or pantothenic acid is readily distributed again substantially uniformly over the entire surface of the eye in each eyelid blink, insofar as a certain draining-away effect should occur.

Extremely advantageously therefore the panthenol and/or pantothenic acid act uniformly on the surface of the eye over the entire duration of the treatment. That can advantageously provide that, when conducting the therapy, on the one hand the dosage of panthenol and/or pantothenic acid can be reduced while on the other hand the duration of the treatment can be shortened.

It is preferred if the amount of panthenol and/or pantothenic acid is about 0.5% by weight to 10% by weight, preferably about 20% by weight to 5% by weight, with respect to the total weight of the pharmaceutical composition. An amount of about 3% by weight with respect to the total weight of the pharmaceutical composition has proven to be highly suitable.

In accordance with a preferred embodiment the panthenol is present in the form of D-(+)-2,4-dihydroxy-N-(3-hydroxypropyl)-3,5-dimethylbutyramide. That dextrorotatary D-configuration is also referred to as dexpanthenol.

In accordance with a further preferred embodiment of the invention the pantothenic acid is in the form of a water-soluble salt, preferably sodium pantothenate or calcium pantothenate.

Extremely advantageously dexpanthenol has water-binding properties. That advantageously supplements the water-binding properties of hyaluronic acid and/or hyaluronate. In addition, the wound healing-promoting effects of hyaluronic acid and/or hyaluronate and pantothenic acid and/or panthenol, in particular dexpanthenol, in dealing with epithelium lesions of the corneal epithelium, supplement each other in a manner which is not understood hitherto.

It is further preferred that the pharmaceutical composition is in the form of a solution, suspension, emulsion, gel, ointment, paste, powder, granules or a tablet which can preferably be used directly on the eye or applied to the surface of the eye.

In accordance with a preferred embodiment the pharmaceutical composition is in the form of a solution so that it can be applied for example in the form of eye drops or an eye spray to the surface of the cornea of the eye.

It will be appreciated that it is possible for the pharmaceutical composition according to the invention to be in the form of a solid which prior to application is firstly dissolved in an aqueous solution such as for example a buffer solution. After dissolution of a solid, for example in an aqueous buffer solution, that solution is subjected to sterile filtering and then applied to the cornea as an eye spray or eye drops. Preferably, the solid and the solvent are already in sterile form when stored separately so that sterile filtration after production of the solution is not required. Thus, the user can apply the pharmaceutical composition directly after making up the mixture or solution.

When preparing the pharmaceutical composition in the form of a solid, such as for example a powder, particles, granules or a tablet the pharmaceutical composition according to this invention preferably includes panthenol, pantothenic acid and/or salts of pantothenic acid, which can be readily dissolved in aqueous solutions, as well as hyaluronic acid which is very soluble in water and/or sodium hyaluronate which is very soluble in water.

Preferably the pharmaceutical composition according to the invention is in sterile form in single-dose or multi-dose receptacles.

Preferably for storage and delivery of a preserving agent-free, pharmaceutical composition in accordance with the invention, use is made of the COMOD® system described in “PTA heute” 1996, No 12, pages 1230-1232, which permits sterile storage and multiple delivery of the pharmaceutical composition according to the invention. It will be appreciated that it is also possible to use conventional single-dose containers which are thrown away after use.

For application of the pharmaceutical composition to the nasal mucous membrane, it is possible to use for example the conventionally known spray receptacles. For example it is possible to use the above-mentioned COMOD® system. The 3K-system (3-Chamber system) described in Deutsche Apotheker Zeitung 139, No 46, pages 48-51, 18th November 1999, has also proven to be very suitable. The pharmaceutical composition can also be dripped into the nose using a pipette.

When using hyaluronic acid and/or hyaluronate in the pharmaceutical composition according to the invention the pharmaceutical composition is preferably prepared free from preserving agent.

Preserving agents can damage the pre-corneal tear film and lead to a reduction in the number of microvilli and microplicae of the surface cornea epithelium cells. In particular the wide-spread benzalkonium chloride has a great damage potential. In regard to the desired therapy of irritation of the eye induced by the Sicca syndrome, it is advantageous to avoid any further irritation and/or damage to the eye by the addition of preserving agents.

In principle the pharmaceutical composition according to the invention can also be introduced into the conjunctival sac in the form of eye tablets. The eye tablet quickly dissolves under the action of tear fluid.

However application of the pharmaceutical composition to the eye in the form of eye drops is preferred.

When preparing the pharmaceutical composition in the form of eye ointments or eye gels or ointments or gels for use in the nose, the active substance are prepared for example in Vaseline or paraffin with and without the addition of emulsifier such as for example cholesterol, wool wax, wool wax alchohols, cetanol, and so forth.

The object of the invention is further attained by the use of a pharmaceutical composition according to one of claims 1 to 8 for the treatment of ophthalmological and/or rhinological malfunctions.

Preferably the pharmaceutical composition according to one of claims 1 to 8 is used for the treatment of ophthalmological malfunctions which are linked to disturbances to wetting of the cornea of the eye.

Further preferably the pharmaceutical composition is used for the treatment of allergic rhinoconjunctivitis, atopic keratoconjunctivitis, allergic keratoconjunctivitis, gigantopapillary conjunctivitis, conjunctivitis vernalis, episcleritis such as for example episcleritis periodica, episcleritis partialis fugax, scleritis, tenonitis, Sjögren syndrome or hybrid forms thereof.

Preferably the pharmaceutical composition according to one of claims 1 to 8 is used for the treatment of rhinological malfunctions which are linked to drying-out phenomena in respect of the nasal mucous membrane.

Further preferably the pharmaceutical composition is used for the treatment of chronic rhinitis, rhinitis sicca, rhinitis sicca anterior or hybrid forms thereof.

The pharmaceutical composition according to the invention can further extremely advantageously be used after operative interventions, for example an operation on the septum of the nose. An application of the composition according to the invention prevents drying-out of the nasal mucous membrane and at the same time promotes re-epithelialisation of the nasal mucous membrane. The pharmaceutical composition according to the invention can also be used after operative interventions on the eye.

As both the eye and also the nose are very important sense organs for the human being, the pharmaceutical composition according to the invention represents a significant advance in the field of ophthalmology and rhinology.

### EXAMPLE

Pharmaceutical Composition for Ophthalmological and Rhinological Application

50 mg/ml of dexpanthenol
1.55 mg/ml of hyaluronic acid, molecular weight: 1.5×10⁶-3.5×10⁶ Daltons
2 mg/ml of sodium citrate
Addition of 1% aqueous citric acid solution until pH of 7.0-pH of 7.4 is reached
Addition of water for injection purposes ad 1 ml

1. Use of panthenol and/or pantothenic acid and hyaluronic acid and/or hyaluronate and optionally additional pharmaceutical auxiliary agents for the production of a pharmaceutical composition for the treatment of ophthalmological and/or rhinological malfunctions.

2. Use according to claim 1 characterised in that the amount of hyaluronic acid and/or the amount of hyaluronate is about 0.005% by weight to about 5% by weight, preferably about 0.01% by weight to about 1% by weight, in each case with respect to the total weight of the pharmaceutical composition.

3. Use according to claim 1 or claim 2 characterised in that the hyaluronic acid and/or the hyaluronate has a molecular weight which is in a range of about 50,000 to about 10,000,000 Daltons, preferably about 250,000 to about 5,000,000 Daltons.

4. Use according to one of claims 1 to 3 characterised in that the hyaluronate is sodium hyaluronate.

5. Use according to one of claims 1 to 4 characterised in that the amount of panthenol and/or pantothenic acid is about 0.5% by weight to 10% by weight, preferably about 2% by weight to 5% by weight with respect to the total weight of the pharmaceutical composition.

6. Use according to one of claims 1 to 5 characterised in that the panthenol is in the form of dexpanthenol.

7. Use according to one of claims 1 to 5 characterised in that the pantothenic acid is in the form of a water-soluble salt, preferably sodium pantothenate or calcium pantothenate.

8. Use according to one of claims 1 to 7 characterised in that the pharmaceutical composition is in the form of a solution, suspension, emulsion, gel, ointment, paste, powder, particles, granules or a tablet.

9. Use according to one of claims 1 to 8 characterised in that the ophthalmological malfunction is linked to disturbances to wetting of the cornea and conjunctiva of the eye.

10. Use according to one of claims 1 to 9 characterised in that the ophthalmological malfunction is selected from the group consisting of allergic rhinoconjunctivitis, atopic keratoconjunctivitis, allergic keratoconjunctivitis, gigantopapillary conjunctivitis, conjunctivitis vernalis, episcleritis, scleritis, tenonitis, Sjögren syndrome and hybrid forms thereof.

11. Use according to one of claims 1 to 8 characterised in that the rhinological malfunction is linked to drying-out phenomena of the nasal mucous membrane.

12. Use according to one of claims 1 to 8 or 11 characterised in that the rhinological malfunction is selected from the group which consists of chronic rhinitis, rhinitis sicca and hybrid forms thereof.
